(19) United States
(12) Patent Application Publication
(10) Pub. No.: US 2026/0185114 A1
(43) Pub. Date: Jul. 2, 2026

(54) METHOD FOR ENHANCING REGENERATION EFFICIENCY USING IMMATURE HEMP EMBRYO

(71) Applicant: NULLA BIO INC., Gyeongsangnam-do (KR)

(72) Inventors: JINHEE JEONG, Gyeongsangnam-do (KR); HYUN JOO SONG, Gyeongsangnam-do (KR); EUNYOUNG JEON, Gyeongsangnam-do (KR)

(21) Appl. No.: 18/857,939

(22) PCT Filed: Sep. 5, 2024
(86) PCT No.: PCT/KR2024/013377

§ 371 Date: Oct. 18, 2024

(30) Foreign Application Priority Data
Sep. 8, 2023 (KR) 10-2023-0119701

(51) Int. Cl. C12N 15/82; C07K 14/415

(52) U.S. Cl. CPC C12N 15/8262; C07K 14/415; C12N 15/8205; C07K 2319/00; C12N 2800/10

(57) ABSTRACT

A recombination vector for enhancing regeneration efficiency of hemp includes a cassette for expressing GRF3 (Growth-regulating factor 3)-GIF1 (GRF-interacting factor 1) chimeric protein or PLT5 (PLETHORA 5) protein. A method for enhancing regeneration efficiency of hemp includes transforming hemp plant cells with the recombination vector. The method may use immature hemp embryo.

### REFERENCE TO AN ELECTRONIC SEQUENCE LISTING

A sequence listing electronically submitted on Oct. 18, 2024 as a XML file named 20241018_S43824GR08_TU_SEQ.XML, created on Oct. 18, 2024 and having a size of 34,696 bytes, is incorporated herein by reference in its entirety.

### BACKGROUND

### 1. Technical Field

The present invention relates to a method for enhancing regeneration efficiency using immature hemp embryo. More specifically, it concerns a method for enhancing the efficiency when regenerating hemp plants in vitro or developing transgenic hemp plants using Agrobacterium.

The present invention was achieved with the support of the Project for Promoting Research Industry of Commercialization Promotion Agency for R&D Outcomes (Foundation), South Korea (Project Number: 2022-Customized Research Startup-Public-02-SB1-01).

### 2. Background Art

In modern life sciences, the development of transformation and tissue culture techniques for hemp or Cannabis sativa has emerged as one of the important research topics. Tissue culture of hemp is a technique that involves culturing tissue explants or cells under appropriate conditions to produce a large amount of hemp plants. This technique is crucial for maintaining consistent quality in hemp plants that are used for hybrid breeding. Additionally, transformation technology is considered to be essential for modifying specific traits or promoting the desired growth and metabolic pathways of hemp plants. In this regard, the regeneration of hemp plays the most important role in both tissue culture and transformation processes. However, an efficient and stable method for transforming hemp plants has not yet been established.

The present invention aims to develop a method and techniques for enhancing the regeneration efficiency of hemp and to determine whether the regeneration efficiency is enhanced in the tissue culture and transformation processes of hemp.

Meanwhile, International Patent Publication No. WO2022/115850 discloses a culture medium composition for micropropagation of auto-flowered hemp plants, titled “TISSUE CULTURE OF AN AUTOFLOWER CANNABIS PLANT,” and U.S. Patent Publication No. 2023-0044740 discloses “IN VITRO DIRECT REGENERATION OF POLYPLOID CANNABIS PLANTS.” However, there is no description regarding the “method for enhancing regeneration efficiency using immature hemp embryo” of the present invention, which involves the timing of obtainment of immature hemp embryo, adjusting conditions for co-cultivation with Agrobacterium for transformation, and use of the GRF3-GIF1 chimeric protein encoding sequence derived from hemp with miR396 resistance to enhance the tissue culture and regeneration efficiency.

### SUMMARY

The present invention was devised in response to the aforementioned needs. The inventors of the present invention aimed to develop, for having a new hemp variety using transformation, a method for enhancing regeneration efficiency of hemp explants. To achieve this, the inventors of the present invention tested the conditions of immature embryo required for obtaining hemp explants and the temperature conditions or the like during co-cultivation with Agrobacterium. As a result, a method that allows for the obtainment of regenerated hemp plants with high-frequency by enhancing the regeneration efficiency of hemp tissue explants was found, and the present invention is completed accordingly.

To solve the problems that are described above, the present invention provides a recombination vector for enhancing regeneration efficiency of hemp, which comprises a cassette for expressing GRF3 (Growth-regulating factor 3)-GIF1 (GRF-interacting factor 1) chimeric protein or PLT5 (PLETHORA 5) protein.

The present invention further provides a method for enhancing regeneration efficiency of hemp, which includes transforming hemp plant cells with the above-mentioned recombination vector.

The present invention further provides a method for producing a transgenic hemp plant with enhanced regeneration efficiency including: transforming hemp plant cells with the above-mentioned recombination vector; and regenerating hemp from the transformed hemp plant cells.

The present invention still further provides a composition for enhancing regeneration efficiency of hemp, which comprises as an effective component a recombination vector comprising a sequence encoding GRF3-GIF1 chimeric protein with the amino acid sequence of SEQ ID NO: 2, or a sequence encoding PLT5 protein with the amino acid sequence of SEQ ID NO: 11.

The present invention provides various methods and systems aimed at enhancing regeneration efficiency of the techniques not only for tissue culture but also for transformation of hemp. It is expected that use of the method of the present invention helps maintaining the hybrid hemp varieties with consistent trait characteristics. Furthermore, with enhancement of the regeneration efficiency of hemp transformation methods, which have not yet been reliably established, the present invention can be effectively utilized across various hemp-related industries and research fields, such as medicine, agriculture, and life sciences research.

### BRIEF DESCRIPTION OF THE DRAWINGS

FIG. 1 is a schematic diagram showing the process of obtaining an immature embryo from hemp plant.

FIG. 2 is a photographic image showing an immature embryo according to the days of embryo rescue (i.e., days after pollination) after hemp female flowers were pollinated by male flowers.

FIG. 3 is a photographic image of a test subject 10 days after the transformation with RUBY using an immature embryo of 13, 14, 17, and 21 DAP.

FIG. 4 is a schematic diagram showing a method of cutting the shoot apical meristem including cotyledons from an immature embryo obtained after embryo rescue, in which the cutting was made using a surgical knife.

FIG. 5A is a photographic image showing the part cut from an immature embryo, in which the cut part was indicated with a red dotted line, FIG. 5B is a photographic image showing the hypocotyl explants from immature embryo to be used in the experiment, in which explants are indicated with a white circle, FIG. 5C is a photographic image of a new shoot being regenerated 10 days after obtaining the hypocotyl explants from the immature embryo, and FIG. 5D is a photographic image of a new shoot being regenerated 14 days after obtaining the hypocotyl explants from the immature embryo.

FIG. 6 shows the results of comparing the regeneration efficiency of hemp hypocotyl explants according to the period of culturing immature embryo (AR; after rescue) between the hemp varieties of Afghani kush and Chung-sam.

FIG. 7 shows the results of comparing the regeneration efficiency of hemp hypocotyls according to temperature conditions during co-cultivation of hemp hypocotyl explants and Agrobacterium.

FIG. 8 shows the results of comparing the regeneration efficiency of hemp hypocotyls when the GRF3-GIF1 chimeric protein encoding sequence derived from hemp with miR396-resistance was introduced.

FIG. 9A is a map of the pLSL.R.Ly-hCas9-GFP recombination vector used in the present invention, FIG. 9B is a map of the pLSL.R.Ly-hCas9-GRF3-GIF1 recombination vector used in the present invention, and FIG. 9C is a map of the pLSL.R.Ly-hCas9-PLT5 recombination vector used in the present invention.

FIG. 10 shows the results of comparing the regeneration efficiency of hemp hypocotyls when the hemp-derived PLT5 protein encoding sequence was introduced.

FIG. 11A is a photographic image taken immediately after transforming, by the method of Example 5, hypocotyl explants obtained according to the method of Example 4 from the immature embryo of the hemp variety Afghani kush, which has been obtained at 21 DAP according to Example 2 of the present invention, with Agrobacterium containing recombination vector pLSL.R.Ly-hCas9-GFP, and FIG. 11B is a photographic image taken 17 days after the transformation (17 DAI).

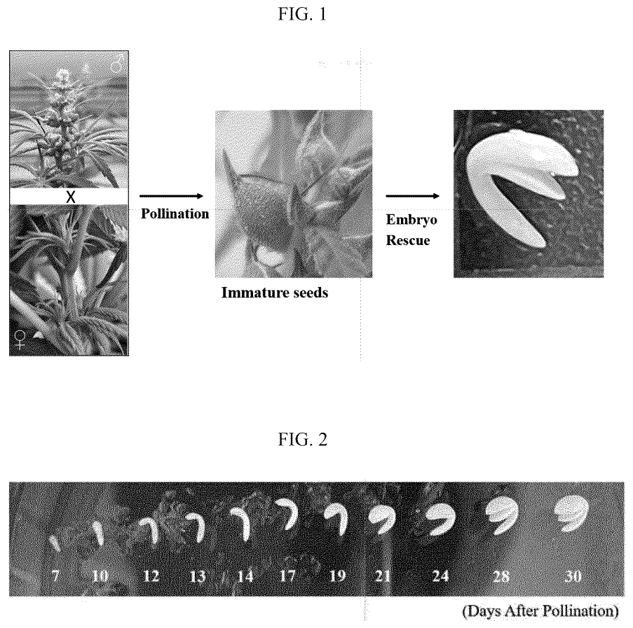

### DETAILED DESCRIPTION

To achieve object of the present invention, the present invention provides a recombination vector for enhancing regeneration efficiency of hemp, which comprises a cassette for expressing GRF3 (Growth-regulating factor 3)-GIF1 (GRF-interacting factor 1) chimeric protein or PLT5 (PLETHORA 5) protein.

In the present invention, the term “recombination” refers to cells that replicate or express heterologous nucleic acids, or cells that express peptides, heterologous peptides, or proteins encoded by heterologous nucleic acids. Recombinant cells can express genes or gene constructs that are not found in the natural form of the cell, either in sense or antisense form. Additionally, recombinant cells can express genes found in natural cells, but those genes are modified and reintroduced into the cells through artificial means.

In the present invention, the term “vector” refers to DNA fragment(s) or nucleic acid molecule(s) used to deliver genetic material into cells. Vectors are capable of replicating DNA and can reproduce independently within host cells. The term “delivery vehicle” is commonly used interchangeably with “vector.”

In the present invention, the term “expression cassette” refers to a nucleic acid sequence that includes one or more genes and sequences that regulate their expression, such as combination of various cis-acting transcription regulatory elements. The expression cassette of the present invention contains the following three main components: i) promoter, ii) a second polynucleotide which may be also referred to as “coding polypeptide” or “coding sequence”, which is operably linked to the promoter and transcribed in accordance with the guidance by the promoter when the expression cassette is introduced to a cell, and iii) terminator polynucleotide (also referred to as a transcription terminator) located immediately downstream of the second sequence and signals the termination of transcription.

The promoter may be one of the promoters suitable for transformation, such as the CaMV 35S promoter, actin promoter, ubiquitin promoter, pEMU promoter, MAS promoter, histone promoter, or Clp promoter, but it is not limited to them. The term “promoter” refers to a DNA region upstream of the structural gene and is a DNA molecule to which RNA polymerase binds to initiate transcription. A “plant promoter” is a promoter that can initiate transcription in plant cells. A “constitutive promoter” is a promoter that is active under most environmental conditions, developmental stages, or cell differentiation. In the present invention, the use of a constitutive promoter may be preferred, and thus the use of a constitutive promoter does not limit the possibility of selection.

In the recombination vector of the present invention, a conventional terminator can be used, and examples of which include the nopaline synthase terminator, rice α-amylase RAmy1 A terminator, octopine gene terminator from Agrobacterium tumefaciens, phaseoline terminator, and the rrnB1/B2 terminator from Escherichia coli, but it is not limited to them. Regarding the necessity of terminators, it is generally known that terminator region can enhance the certainty and efficiency of gene transcription in plant cells. Therefore, the use of a terminator is highly preferred in the context of the present invention.

In the recombination vector of the present invention, the GRF3-GIF1 chimeric protein includes the protein having the amino acid sequence represented by SEQ ID NO: 2 and functional equivalents of this protein. A “functional equivalent” refers to a protein that, as a result of additions, substitutions, or deletions of amino acids, exhibits at least 70% sequence identity, preferably 80% or more, more preferably 90% or more, and even more preferably 95% or more with the amino acid sequence represented by SEQ ID NO: 2, and demonstrates substantially equivalent physiological activity to the protein represented by SEQ ID NO: 2. “Substantially equivalent physiological activity” refers to the activity of enhancing regeneration efficiency of hemp plants.

Further, in the recombination vector of the present invention, the PLT5 protein includes the protein having the amino acid sequence represented by SEQ ID NO: 11 and functional equivalents of this protein.

Further, in the present invention, the sequence encoding the GRF3-GIF1 chimeric protein may consist of the nucleotide sequence represented by SEQ ID NO: 1, but is not limited thereto. Additionally, homologs of this nucleotide sequence are included within the scope of the present invention. Specifically, the coding sequence may include nucleotide sequences that have at least 70% sequence identity, more preferably 80% or more, even more preferably 90% or more, and most preferably 95% or more compared to the nucleotide sequence of SEQ ID NO: 1. The “percentage (%) of sequence identity” for a polynucleotide is determined by comparing two optimally aligned sequences within a comparison region, where parts of the polynucleotide sequence in the comparison region may include additions or deletions (i.e., gaps) compared to the reference sequence of the two sequences (not including any addition or deletion).

In the present invention, the nucleotide sequence of SEQ ID NO: 1 specifically includes a hemp-derived sequence encoding GRF3 with miR396 resistance, a linker sequence, and a hemp-derived sequence encoding GIF1, which are arranged in order. The miR396-binding sequence is located within the GRF3 encoding sequence. Specifically, the hemp-derived GRF3 encoding sequence with miR396 resistance spans from the 1^{st} to the 1,314^{th} nucleotide in SEQ ID NO: 1, the hemp-derived GIF1 encoding sequence spans from the 1,327th to the 1,992^{nd} nucleotide in SEQ ID NO: 1, and the miR396-binding sequence is spans from the 597^{th} to the 609^{th} nucleotide in SEQ ID NO: 1.

In the present invention, the sequence encoding PLT5 protein may consist of the nucleotide sequence represented by SEQ ID NO: 10, but is not limited thereto. Additionally, homologs of this nucleotide sequence are included within the scope of the present invention, and specifics are the same as those described in the above.

The recombination vector for enhancing regeneration efficiency of hemp according to the present invention may additionally include, but is not limited to, a cassette for expressing a foreign gene or a gene editing cassette, in addition to the chimeric protein expression cassette.

The foreign gene expression cassette may include a gene encoding a target protein that is intended to be expressed in hemp.

Additionally, the gene editing cassette may include an endonuclease expression cassette and a guide RNA (gRNA) expression cassette, but it is not limited to them.

In the present invention, the term “gene/genome editing” refers to a technology for introducing target-oriented mutations into the genomic nucleotide sequence of cells, including human, animal, and plant cells. This involves techniques that induce deletions, insertions, or substitutions of one or more nucleic acid molecules through DNA cleavage, allowing knock-outs or knock-ins of a specific gene. It can also introduce mutations into non-coding DNA sequences that do not produce any protein. Additionally, “gene editing” can be used interchangeably with “gene correction.”

In the present invention, the endonuclease protein can be one or more selected from the group consisting of Cas9, Cpf1 (also known as Cas12a), TALEN (Transcription Activator-Like Effector Nuclease), ZFN (Zinc Finger Nuclease), and functional analogs thereof. Preferably, it may be an RNA-guided nuclease such as Cas9 or Cpf1, and more preferably, it may be the Cas9 protein, although it is not limited thereto.

In addition, the Cas9 protein may be one or more selected from the group consisting of Cas9 protein derived from Streptococcus pyogenes, Cas9 protein derived from Campylobacter jejuni, Cas9 protein derived from Streptococcus thermophilus, Cas9 protein derived from Staphylococcus aureus, Cas9 protein derived from Neisseria meningitidis, Cas9 protein derived from Pasteurella multocida, Cas9 protein derived from Francisella novicida and the like, although it is not limited thereto. The Cas9 protein or its genetic information can be obtained from public databases such as GenBank at the NCBI (National Center for Biotechnology Information). The Cas9 gene information can be used as-is from published sequences or can be optimized for the codon usage of the target transgenic organism, although it is not limited thereto.

The Cas9 protein is an RNA-guided DNA endonuclease enzyme that induces double-stranded DNA breaks. For Cas9 to accurately bind to and cleave the target DNA sequence, a short sequence of three nucleotides known as PAM (Protospacer Adjacent Motif) must be present adjacent to the target nucleotide sequence, and the Cas9 protein recognizes and cleaves the DNA between the third and fourth base pairs from the PAM sequence (NGG).

The CRISPR/Cas9 system is a gene editing method that introduces double-strand breaks at specific locations within a target gene. This induces insertion-deletion (InDel) mutations through the non-homologous end joining (NHEJ) repair mechanism, which results from the imperfect repair of the DNA breaks.

In addition, the term “guide RNA” in the present invention refers to a short single-stranded RNA, which is specific to the target DNA among the base sequences encoding the target gene, and it refers to a ribonucleic acid that complementarily binds to all or part of the target DNA base sequence and guides an endonuclease protein to the target DNA base sequence. The guide RNA refers to a dual RNA comprising two RNAs, namely, crRNA (CRISPR RNA) and tracrRNA (trans-activating crRNA) as constitutional components; or a single-stranded guide RNA (sgRNA) comprising a first portion including a sequence complementary to all or part of the base sequence in the target gene and a second portion including a sequence interacting with an endonuclease (particularly, RNA-guide nuclease), but both are included in the scope of the present invention without any limitation. The guide RNA can be produced and used according to a technique known in the pertinent art, taking into consideration the type of endonuclease used together or the microorganism from which the endonuclease is derived. The guide RNA may be adjacent to a PAM (protospacer adjacent motif) site and may include a sequence complementary to a 10 to 20 bp base sequence of the DNA to be edited, but is not limited thereto.

The guide RNA may be, but is not limited to, a guide RNA transcribed from a plasmid template, a guide RNA transcribed in vitro (e.g., an oligonucleotide duplex), or a synthetic guide RNA.

In addition, the recombination vector for enhancing regeneration efficiency of hemp according to the present invention may be composed of the GRF3-GIF1 chimeric protein or PLT5 protein expression cassette; and the foreign gene expression cassette or gene correction cassette, either as separate plasmids or as a single plasmid, but it is not limited thereto.

The recombination expression vector according to the present invention can be constructed using methods known to those skilled in the pertinent art. The methods include in vitro recombinant DNA techniques, DNA synthesis techniques, and in vivo recombinant technologies. The DNA sequence can be effectively linked to a suitable promoter within the expression vector to induce mRNA synthesis. Additionally, the expression vector may include a ribosome binding site as the translation initiation site and a transcription terminator.

The recombination vector preferably may preferably include one or more selection markers. Those markers are nucleic acid sequences that confer traits which can be generally selected chemically, allowing differentiation between transformed and non-transformed cells. Examples thereof include: herbicide resistance genes, such as those conferring resistance to glyphosate or phosphinothricin; antibiotic resistance genes, such as those providing resistance to kanamycin, hygromycin, chloramphenicol, G418 (geneticin), or bleomycin; and the aadA gene (aminoglycoside-3′-adenyl transferase), but it is not limited thereto.

A preferred example of the recombination vector of the present invention is a Ti-plasmid vector, which, when present in a suitable host such as Agrobacterium tumefaciens, can transfer part thereof, i.e., so-called T-region, to plant cells. Different types of Ti-plasmid vectors (e.g., as described in EP 0 116 718 B1) are used to transfer hybrid DNA sequences into plant genomes to produce new plants. A particularly preferred form of Ti-plasmid vector is the so-called binary vector, as described in EP 0 120 516 B1 and U.S. Pat. No. 4,940,838. Other suitable vectors for introducing the DNA according to the present invention into plant hosts include viral vectors derived from double-stranded plant viruses (e.g., CaMV) and single-stranded viruses or gemini viruses. Examples thereof include incomplete plant virus vectors. The use of such vectors is advantageous, especially when it is challenging to transform appropriately plant hosts.

The present invention further provides a method for enhancing regeneration efficiency of hemp, which includes transforming hemp plant cells with the above-mentioned recombination vector.

As for the method for transporting a recombination vector into a host cell include, when the host cell is a prokaryotic cell, the CaCl₂ method, the Hanahan method (Hanahan, D., J. Mol. Biol., 166:557-580 (1983)), and electroporation, etc. can be carried out. In addition, when the host cell is a eukaryotic cell, gene gun-mediated transformation method (bombardment), Agrobacterium-mediated transformation method, microinjection method, calcium phosphate precipitation method, electroporation method, liposome-mediated transfection method, and DEAE-dextran treatment, etc. can be used for introducing the vector into the host cell, but is not limited thereto.

More specifically, the method for enhancing regeneration efficiency of hemp according to the present invention may include the following:
obtaining an immature embryo from a hemp plant,
cutting off a shoot apical meristem including cotyledons from the obtained immature embryo; and
co-cultivating the shoot apical meristem including cotyledons with Agrobacterium tumefaciens transformed with the recombination vector according to the present invention,
but it is not limited thereto.

In one embodiment of the method for enhancing regeneration efficiency of hemp according to the present invention, the immature embryo is preferably an embryo obtained 17 to 24 days after pollination. More preferably, the immature embryo is an embryo obtained 20 to 22 days after pollination, and even more preferably, 21 days after pollination, but it is not limited thereto.

In the present invention, in order to obtain an immature embryo from hemp, a hemp plant or seeds were grown at 22 to 28° C. under long-period light conditions for more than 3 to 6 weeks, and then the hemp plant was transferred to short-period light conditions to induce flowering. At this time, when a PCR test using a sex determination marker was performed or when flower buds (immature flowers) were generated, male and female trees were separated from each other and grown in isolation. Afterwards, when the male hemp trees bloomed and produced pollen, female hemp flowers or female trees were pollinated and, starting basically from the pollination day, immature embryos were obtained before the embryos were fully mature.

Furthermore, in one embodiment of the method for enhancing regeneration efficiency of hemp according to the present invention, the co-cultivation step is preferably conducted under dark conditions at a temperature ranging from 4° C. to 25° C. for 2 to 4 days. More preferably, the co-cultivation is carried out at a temperature between 16° C. and 25° C. for 3 days, although it is not limited thereto.

With regard to the method for enhancing regeneration efficiency of hemp according to the present invention, it may further include a step of transferring the shoot apical meristem including cotyledons which has been co-cultivated with Agrobacterium tumefaciens to a regeneration medium and further cultivating at 22° C. to 25° C. for 7 to 14 days, but it is not limited thereto.

The present invention further provides a method for producing a transgenic hemp plant with enhanced regeneration efficiency including the following:
transforming hemp plant cells with the above-mentioned recombination vector of the present invention; and
regenerating hemp from the transformed hemp plant cells.

In the production method according to the present invention, the recombination vector is as described in the above.

With regard to the production method according to one embodiment of the present invention, the recombination vector may be a plasmid including an expression system capable of inserting a DNA encoding a gRNA specific to a target DNA for gene correction (editing) in a hemp plant and a nucleic acid sequence encoding an endonuclease protein and expressing the same in a hemp plant cell, but it is not limited thereto. The plasmid includes elements required for the expression of a target gene, and it may include a replication origin, a promoter, an operator, a transcription terminator, etc., and may further include an appropriate enzyme site (e.g., a restriction enzyme site) for introduction into the genome of a host cell and/or a selection marker for determining successful introduction into a host cell, and/or a ribosome binding site (RBS) for translation into a protein, and/or a transcriptional regulatory element, etc.

In addition, the production method of the present invention includes a step of regenerating a transformed hemp plant from the transformed hemp plant cells. As for the method for regenerating transformed plant, any method known in the pertinent art can be utilized.

The present invention still further provides a composition for enhancing regeneration efficiency of hemp, which comprises as an effective component a recombination vector comprising a sequence encoding GRF3-GIF1 chimeric protein with the amino acid sequence of SEQ ID NO: 2, or a sequence encoding PLT5 protein with the amino acid sequence of SEQ ID NO: 11. Since the composition includes, as an effective component, a sequence encoding the GRF3-GIF1 chimeric protein with the amino acid sequence of SEQ ID NO: 2 or a sequence encoding PLT5 protein with the amino acid sequence of SEQ ID NO: 11, both having the effect of enhancing regeneration efficiency of hemp plant tissues, the regeneration efficiency of hemp plant can be enhanced by transforming hemp plant cells with a recombination vector which contains the sequence encoding the GRF3-GIF1 chimeric protein or the PLT5 protein.

Hereinbelow, the present invention is explained in greater detail in view of Examples. However, the following Examples are given only for exemplification of the present invention and it is evident that the scope of the present invention is not limited by them.

### EXAMPLES

### Example 1. Cloning of Gene Expressing GRF3-GIF1 Chimeric Protein Derived from Hemp with miR396-Resistance

To obtain the gene which encodes the GRF3 (Growth-regulating factor 3)-GIF1 (GRF-interacting factor 1) chimeric protein derived from hemp with miR396-resistance (SEQ ID NO: 1), which will be introduced to enhance the regeneration efficiency, site-directed mutagenesis was carried out using PCR (polymerase chain reaction) with hemp cDNA from the Chung-sam variety as a template. Specifically, the cloning was performed following the technique described by Hemsley, Anne, et al. in Nucleic Acids Research (1989, 17 (16): 6545-51).

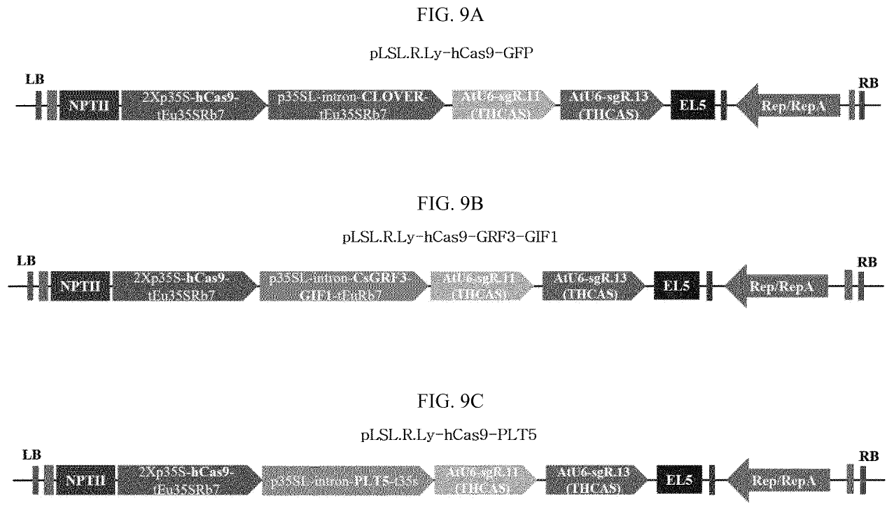

Afterwards, bean yellow dwarf virus (BeYDV)-derived replicon pLSL.R.Ly was used as a recombination vector backbone for gene expression in hemp plant (Vu, Tien Van, et al., Plant Biotechnol J. 2020, 18 (10): 2133-2143), and between the LB (left border) and RB (right border) sequences, a recombination vector pLSL.R.Ly-hCas9-GFP (FIG. 9A) containing an antibiotic marker expression cassette; green fluorescent protein (GFP) expression cassette; an endonuclease expression cassette and a gRNA expression cassette targeting the hemp-derived THCAS (tetrahydrocannabinolic acid synthase) gene (GenBank: MN422084.1) were constructed, and, between the LB and RB sequences, recombination vector pLSL.R.Ly-hCas9-GRF3-GIF1 (FIG. 9B) containing an antibiotic marker expression cassette; an expression cassette for GRF3-GIF1 chimeric protein from hemp with miR396-resistance; an endonuclease expression cassette and a gRNA expression cassette targeting the hemp-derived THCAS gene were constructed by Golden Gate cloning which uses BsaI, a Type IIS restriction enzyme (Engler et al., PLOS One. 2008, 3 (11): e3647).

In the vector map of FIG. 9, 2Xp35S-hCas9-tEu35SRb7 (SEQ ID NO: 3) corresponds to the endonuclease expression cassette, p35SL-intron-CLOVER-tEu35SRb7 (SEQ ID NO: 4), p35SL-intron-CsGRF3-GIF1-tEuRb7 (SEQ ID NO: 5) and p35SL-intron-PLT5-135s (SEQ ID NO: 12) correspond to GFP, GRF3-GIF1, and PLT5 expression cassettes, respectively, and AtU6-sgR.11 (THCAS) (SEQ ID NO: 8) and AtU6-sgR.13 (THCAS) (SEQ ID NO: 9) correspond to gRNA expression cassettes targeting the hemp-derived THCAS gene and they are the cassettes that express the gRNAs shown in Table 1 below, respectively.

TABLE 1
gRNA targeting THCAS gene derived from hemp

| | Nucleotide Sequence (5′→3′) | SEQ ID NO: |
|---|---|---|
| gRNA11 | ACCAATTCCAGAAACTGCAA | 6 |
| gRNA13 | cccttacggtggtataatgg | 7 |

Agrobacterium tumefaciens EHA-105 were transformed with the recombination vector by using an electroporation method.

### Example 2. Method for Obtaining Immature Embryo from Hemp Plant

The present invention was carried out with the narcotics handler (academic researcher) license (Identification Number L00202725).

To obtain immature embryos in the present invention, hemp was grown under long-day conditions (16 hours light/8 hours dark) during the flowering period in a greenhouse. At the fifth week, the plants were switched to short-day conditions (12 hours light/12 hours dark) to induce flowering. At that time, male and female plants were separated based on sex-determination markers using PCR (Faux, Anne-Michelle, et al., Euphytica 2014, 196:183-197), and, upon the flowering from each tree, pollen from the male flowers was used to fertilize the pistils of the female flowers. The period from the day of pollination to embryo rescue was referred to as Days After Pollination (DAP). In the present invention, embryo rescue was carried out at 7, 10, 12, 13, 14, 17, 19, 21, 24, 28, or 30 DAP, and the state of each embryo was examined at the time of the embryo rescue (FIG. 2).

### Example 3. Method for Culturing Immature Embryos

In the present invention, the immature embryos obtained through embryo rescue were cultured at 25° C. on a solid medium (MS basic medium, 1.5% sucrose, 5% agar, pH 5.8) until the test. The period of culture after embryo rescue of immature embryos was referred to as After Rescue (AR).

### Example 4. Method for Obtaining Hypococtyl Explants from Hemp Seedling or Immature Embryos

After embryo rescue, by using a razor blade, shoot apical meristem including cotyledons was cut from the immature embryos or hemp seedlings cultured for several days to obtain hypococtyl explants (FIG. 4).

### Example 5. Method of Transforming Hemp Using Agrobacterium

The immature embryo's hypocotyl explants obtained from above Example 4 were immersed for 40 to 50 minutes in an inoculation medium (1/2 MS basic medium, 2% sucrose, 1% glucose, 200 μM acetosyringone, pH 5.2) in which Agrobacterium tumefaciens containing the recombination plant expression vector is suspended. The explants were then co-cultivated under dark conditions.

The Agrobacterium tumefaciens transformed with the recombinant plant expression vector was cultured in liquid LB medium containing 20 mg/L rifampicin, 25 mg/L gentamicin, and 50 mg/L kanamycin at 28° C., 180 rpm for 16 hours. After that, the culture was diluted 1:10 with 30 mL of fresh medium and further incubated at 28° C., 180 rpm for 4 to 6 hours. The Agrobacterium (OD₆₀₀=0.6 to 0.8) was collected by centrifugation at 3,500 rpm and resuspended in 30 mL of liquid inoculation medium containing 200 μM acetosyringone (AS) (pH 5.2; Wu et al., Plant Methods 2014, 10:19). The suspension was activated at 28° C., 180 rpm for 1 hour prior to use for hemp transformation.

### Example 6. Determination of Regeneration Efficiency from Hemp Hypococtyl Explants

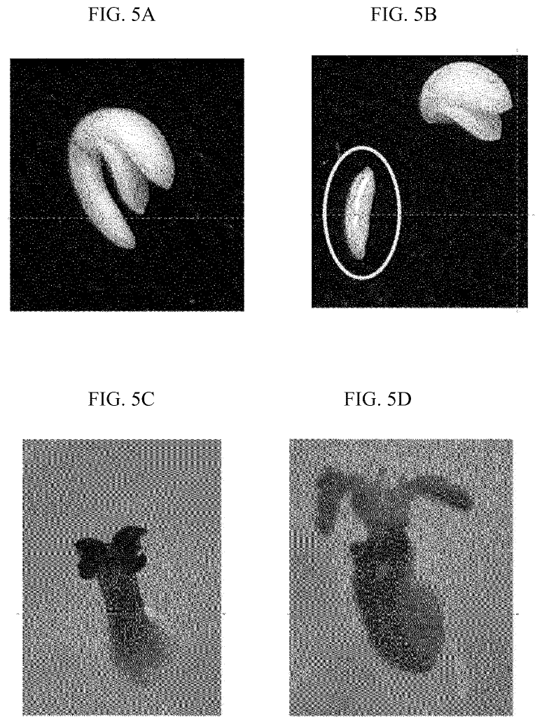

The hypocotyl explants obtained from the above Example 4 were cultured in MS basic medium or regeneration medium containing 0.2 mg/L auxin (e.g., 1-Naphthaleneacetic acid (NAA)) and 0.4 mg/L cytokinin (e.g., Thidiazuron (TDZ)), while the hypocotyl explants co-cultivated in the above Example 5 were cultured in MS basic medium containing 300 mg/L timentin or regeneration medium with 0.2 mg/L auxin and 0.4 mg/L cytokinin at 22 to 25° C. for 7 to 14 days. After that, the number of explants producing new shoots was counted to determine the regeneration efficiency (FIG. 5).

### Example 7. Determination of Growth of Transgenic Hemp Hypococtyl Explants by DAP

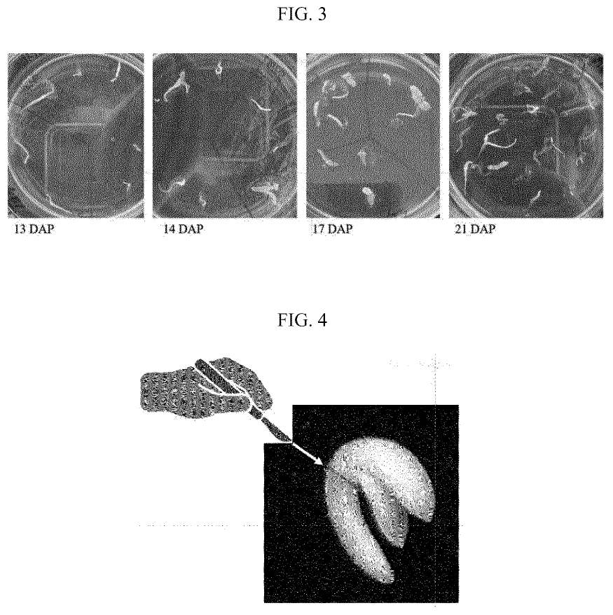

The immature embryos obtained at 13, 14, 17, or 21 DAP in the above Example 5 were cultured for 3 days, followed by the obtainment of hypocotyl explants from those embryos as described in Example 4. Then, the hypocotyl explants were co-cultivated with Agrobacterium containing a vector expressing the RUBY reporter (HE, Yubing, et al., Horticulture Research, 2020, 7:152) by using the method described in Example 5. After 10 days, the hemp explant tissues were examined, and it was found that the best growth performance is observed when the 21 DAP immature embryos are used (FIG. 3).

### Example 8. Determination of Regeneration Efficiency of Hemp Hypococtyl Explants Depending on Different Culture Period of Immature Embryo

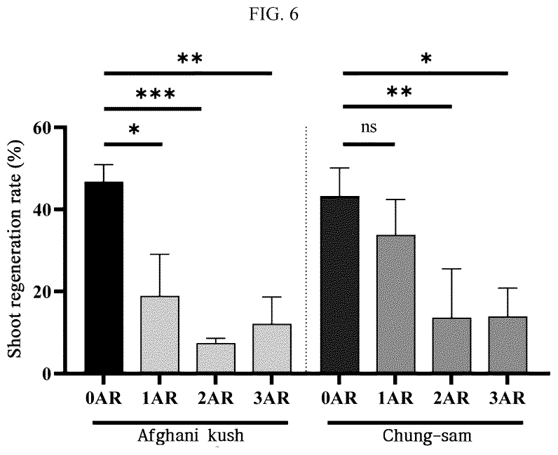

The immature embryos of the hemp varieties Afghani Kush and Chung-sam, which had been obtained at 21 DAP according to the above Example 2, were cultured for 0, 1, 2, or 3 AR (after rescue) periods. Hypocotyl explants were then obtained as described in Example 4. After that, each of the explants was transformed with Agrobacterium containing the recombination vector pLSL.R.Ly-hCas9-GFP. Seven days after co-cultivation by following the method described in Example 5, regeneration efficiency was determined. As a result, it was found that both hemp varieties exhibit the highest regeneration efficiency at 0 AR (FIG. 6).

### Example 9. Determination of Regeneration Efficiency of Hemp Hypococtyl Explants Under Various Co-Cultivation Temperature Conditions

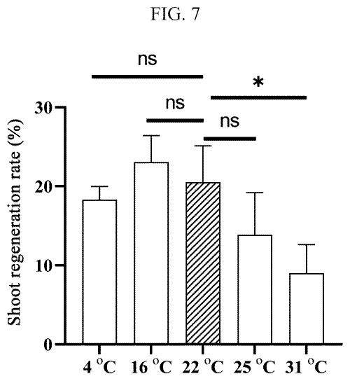

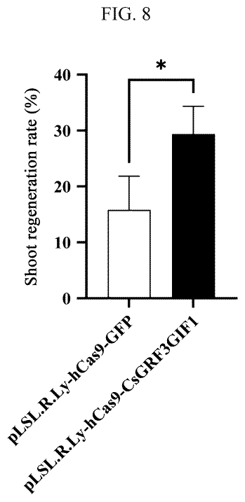

From the immature embryos of the Afghani Kush hemp variety at 21 DAP, which had been obtained at 21 DAP according to the above Example 2, hypocotyl explants were obtained as described in Example 4. According to Example 5, the explants were immersed in a suspension of Agrobacterium containing the recombination vector pLSL.R.Ly-hCas9-GFP and co-cultivated under various temperature conditions. Specifically, the explants were co-cultivated for 3 days in the dark at a temperature of 4° C., 16° C., 22° C., 25° C., or 31° C. After 7 days, the regeneration efficiency was determined. The highest regeneration efficiency was observed at 16° C., with no significant difference compared to temperatures of from 4° C. to 22° C. However, it was found that the regeneration efficiency decreased starting from 25° C. and was notably lower at 31° C. (FIG. 7).

### Example 10. Determination of Regeneration Efficiency of Hemp Hypococtyl According to Introduction of Hemp-Derived Gene which Expresses GRF3-GIF1 Chimeric Protein with miR396-Resistance

From the immature embryos of the Afghani Kush hemp variety at 21 DAP, which had been obtained at 21 DAP according to the above Example 2, hypocotyl explants were obtained as described in Example 4. Then, the hypocotyl explants were transformed using Agrobacterium containing either the recombination vector pLSL.R.Ly-hCas9-GFP or pLSL.R.Ly-hCas9-CsGRF3GIF1, as described in Example 5. After transformation, the explants were co-cultivated at 22° C. in the dark for 3 days. Seven days later, the regeneration efficiency was determined. It was found that explants introduced with the GRF3-GIF1 chimeric protein encoding gene, which is derived from the hemp with miR396-resistance, exhibits better regeneration efficiency compared to those not introduced with GRF3-GIF1 chimeric protein encoding gene (FIG. 8).

### Example 11. Determination of Regeneration Efficiency of Hemp Hypococtyl According to Introduction of Hemp-Derived Gene which Encodes PLT5 Protein

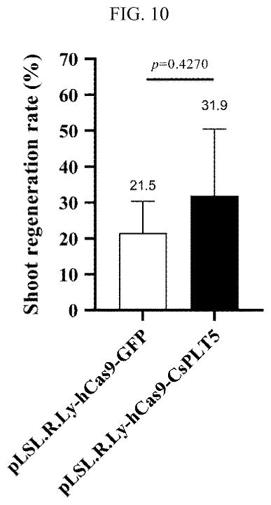

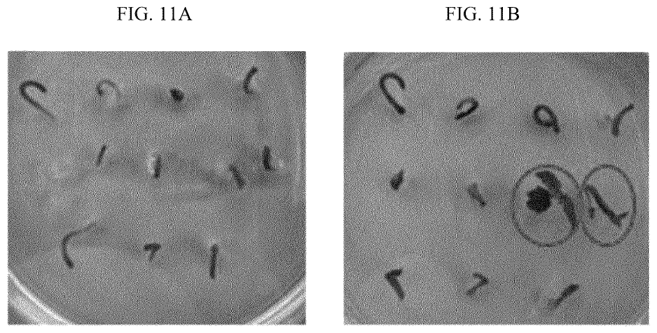

From the immature embryos of the Afghani Kush hemp variety at 21 DAP, which had been obtained at 21 DAP according to the above Example 2, hypocotyl explants were obtained as described in Example 4. Then, the hypocotyl explants were transformed using Agrobacterium containing either the recombination vector pLSL.R.Ly-hCas9-GFP or pLSL.R.Ly-hCas9-PLT5, as described in Example 5. After transformation, the explants were co-cultivated at 22° C. in the dark for 3 days. Seven days later, the regeneration efficiency was determined. It was found that explants introduced with the hemp-derived PLT5 protein encoding gene exhibit better regeneration efficiency compared to those not introduced with PLT5 protein encoding gene (FIG. 10).

The present invention was achieved with the support of the Ministry of SMEs, South Korea and the “Gyeongnam Regional Business Growth-Ladder Support Project” of Gyeongsangnam-do, South Korea.

1. A recombination vector for enhancing regeneration efficiency of hemp, comprising a cassette for expressing GRF3 (Growth-regulating factor 3)-GIF1 (GRF-interacting factor 1) chimeric protein or PLT5 (PLETHORA 5) protein.

2. The recombination vector according to claim 1, wherein it comprises additionally a cassette for expressing a foreign gene or a gene editing cassette.

3. The recombination vector according to claim 2, wherein the cassette for expressing GRF3-GIF1 chimeric protein or PLT5 protein; and the cassette for expressing a foreign gene or gene editing cassette are present as either separate plasmids or a single plasmid.

4. The recombination vector according to claim 1, wherein the GRF3-GIF1 chimeric protein consists of the amino acid sequence of SEQ ID NO: 2 and the PLT5 protein consists of the amino acid sequence of SEQ ID NO: 11.

5. A method for enhancing regeneration efficiency of hemp, the method comprising transforming hemp plant cells with the recombination vector of claim 1.

6. The method according to claim 5, wherein the transforming comprises:
obtaining an immature embryo from a hemp plant,
cutting off a shoot apical meristem including cotyledons from the obtained immature embryo; and
co-cultivating the shoot apical meristem including cotyledons with Agrobacterium tumefaciens transformed with the recombination vector.

7. The method according to claim 6, wherein the immature embryo is obtained 17 to 24 days after pollination.

8. The method according to claim 6, wherein the co-cultivating is cultivating under dark conditions at a temperature of from 4° C. to 25° C. for 2 to 4 days.

9. A method for producing a transgenic hemp plant with enhanced regeneration efficiency, the method comprising:
transforming hemp plant cells with the recombination vector of claim 1; and
regenerating hemp from the transformed hemp plant cells.

10. A composition for enhancing regeneration efficiency of hemp, comprising:
as an effective component, a recombination vector comprising a sequence encoding GRF3 (Growth-regulating factor 3)-GIF1 (GRF-interacting factor 1) chimeric protein with the amino acid sequence of SEQ ID NO: 2, or a sequence encoding PLT5 (PLETHORA 5) protein with the amino acid sequence of SEQ ID NO: 11.


SEQUENCE LISTING (12 sequences)

SEQ ID NO: 1 - DNA, 1992 bp, synthetic construct
  source 1..1992: mol_type = other DNA
atggacttccatctgaagcaatggagaaatcagcaacatgagtcagaacatcaccaacaacattctgcaaagataccaaaacttgttatcgaaacccatcacatgaaacaagacctctcttctacttgttctgaacctcttcctctgtttgtacctgagccaccaaacatcactaaaatgatcacaactggtactctgtctactgattcaactacttctcccactactagaatcccaagaggaggaatgggtagttactttagtttggcccaatggcaagaactagagttacaagctttgatatttaggtacatgttagctggtgctactgttccttctgagcttcttcaacatattaagaaaagctttaatcttcttcatacgacaccgtatttcttgcatcattctcttcaaccttatccccattttcaaccttcccctactttgttgcagtcgggttattggggaaaagcatccatggatccggaacctggtcggtgccggagaacagacgggaagaaatggcggtgttcaagagatgtggtggttggtcaaaagtactgtgagcgccacatgcaccgtggaagaaatcgttctagaaaaccggtcgaaatccccacacccaccaccaccaccaccggcggcggaaggctcgccggggacggcgtcggaagagggactcttgcttctaacaatggtccatctcctttgtcaacgtcatctaatgcagcccatttctcactttcaatgccaacatcctccatagatctcctccatctcaacaaccggagtaacgcttcagatgttaaaaatgaaaataaggacttgtttaaaccccataatgaagtctccggcgacggcaaatcggacggtcacatactccggcacttctttgatgactggccgagatcacttcaagaatcaggaaatgtagaaattaataataataataataataataacaataacaataacaataataggatgaggacgagtccaatgagctcagcctcaagcttatcaatttccataacgggaaacaactcatcatcaacttctgatatttcgttgaagttgtcaactggtggaaacggagaggaaatgggccacccagaagcccattctgatcgtcagcagcagcagtctcagttgggctgggcctcgagtggatgggcttcaaacccaatggcctcaatgggtgggccgctggctgaggccctgcgatcctccaactccaattcatcatctcctactagtgttctccatcagttgcctcctcgttcttctgcctctgacacttgcattgtcagcactgcggccgctgccatgcagcagcacctgatgcagatgcagcccatgatggcagcctattatcccaacaacgtcaccactgatcacattcaacagtatttggatgaaaacaagtcactgatcctgaagattgttgagagccagaattcaggaaaattgggtgaatgtgcagagaaccaagcgaggctgcagcgaaacctcatgtacctggctgctattgctgattctcagccccagcctcctgcaatgcattctcagtatccttcaagtagtattatgcagcctggagcacattacatgcagcagcaccaacaagctcagcagataactcaacaatcactcatggcgggaggctcgtccatgctgtacaaccagcagccatttacagctttgcaacagcaagcactgcacaaccagctcggcatgagctctggtggaaactcgggacttcacatgctgcaaagtgacatgggtagcgctggaggcagtgggcagcagcttggaggtggaggatttcctgatttcagccgtggctctggaggagaagggatgcatagaggaatggctggcggtagtaagcaagatatgggaagttctggatcggctgaaggccgaggagggagctctggaggtgacgggggtgaaaccctttacttaaaatctgccgatgatgggaattaa

SEQ ID NO: 2 - protein, 663 aa, synthetic construct
MDFHLKQWRNQQHESEHHQQHSAKIPKLVIETHHMKQDLSSTCSEPLPLFVPEPPNITKMITTGTLSTDSTTSPTTRIPRGGMGSYFSLAQWQELELQALIFRYMLAGATVPSELLQHIKKSFNLLHTTPYFLHHSLQPYPHFQPSPTLLQSGYWGKASMDPEPGRCRRTDGKKWRCSRDVVVGQKYCERHMHRGRNRSRKPVEIPTPTTTTTGGGRLAGDGVGRGTLASNNGPSPLSTSSNAAHFSLSMPTSSIDLLHLNNRSNASDVKNENKDLFKPHNEVSGDGKSDGHILRHFFDDWPRSLQESGNVEINNNNNNNNNNNNNNRMRTSPMSSASSLSISITGNNSSSTSDISLKLSTGGNGEEMGHPEAHSDRQQQQSQLGWASSGWASNPMASMGGPLAEALRSSNSNSSSPTSVLHQLPPRSSASDTCIVSTAAAAMQQHLMQMQPMMAAYYPNNVTTDHIQQYLDENKSLILKIVESQNSGKLGECAENQARLQRNLMYLAAIADSQPQPPAMHSQYPSSSIMQPGAHYMQQHQQAQQITQQSLMAGGSSMLYNQQPFTALQQQALHNQLGMSSGGNSGLHMLQSDMGSAGGSGQQLGGGGFPDFSRGSGGEGMHRGMAGGSKQDMGSSGSAEGRGGSSGGDGGETLYLKSADDGN

SEQ ID NO: 3 - DNA, 6175 bp, synthetic construct
  source 1..6175: mol_type = other DNA
gaattcaagcttggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgcccaaaaagaaaagaaaagtggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 4 - DNA, 3649 bp, synthetic construct
  source 1..3649: mol_type = other DNA
ggaggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataaggtaaatttctgtgttccttattctctcaaaatcttcgattttgttttcgttcgatcccaatttcgtatatgttctttggtttagattctgttaatcttagatcgaagatgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggagctcatttttacaacaattaccaacaacaacaaacaacaaacaacattacaattacatttacaattatcgatacaatggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcacggccgacaagcagaagaacggcatcaaggctaacttcaagatccgccacaacgttgaggacggcagcgtgcagctcgccgaccactaccagcagaacacccccatcggcgacggccccgtgctgctgcccgacaaccactacctgagccatcagtccgccctgagcaaagaccccaacgagaagcgcgatcacatggtcctgctggagttcgtgaccgccgccgggattacacatggcatggacgagctgtacaagtctagagggccctattctatagtgtcacctaaatgctagagcttaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 5 - DNA, 4663 bp, synthetic construct
  source 1..4663: mol_type = other DNA
gaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataaggtaaatttctgtgttccttattctctcaaaatcttcgattttgttttcgttcgatcccaatttcgtatatgttctttggtttagattctgttaatcttagatcgaagatgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggagctcatttttacaacaattaccaacaacaacaaacaacaaacaacattacaattacatttacaattatcgatacaatggacttccatctgaagcaatggagaaatcagcaacatgagtcagaacatcaccaacaacattctgcaaagataccaaaacttgttatcgaaacccatcacatgaaacaagacctctcttctacttgttctgaacctcttcctctgtttgtacctgagccaccaaacatcactaaaatgatcacaactggtactctgtctactgattcaactacttctcccactactagaatcccaagaggaggaatgggtagttactttagtttggcccaatggcaagaactagagttacaagctttgatatttaggtacatgttagctggtgctactgttccttctgagcttcttcaacatattaagaaaagctttaatcttcttcatacgacaccgtatttcttgcatcattctcttcaaccttatccccattttcaaccttcccctactttgttacagtcgggttattggggaaaagcatccatggatccggaacctggtcggtgccggagaacagacgggaagaaatggcggtgttcaagagatgtggtggttggtcaaaagtactgtgagcgccacatgcaccgtggaagaaatcgttcaagaaagcctgtggaaatccccacacccaccaccaccaccaccggcggcggaagattcgccggggacggcgtcggaagagggactcttgcttctaacaatggtccatctcctttgtcaacgtcatctaatgcagcccatttctcactttcaatgccaacatcctccatagatctcctccatctcaacaaccggagtaacgcttcagatgttaaaaatgaaaataaggacttgtttaaaccccataatgaagtctccggcgacggcaaatcggacggtcacatactccggcacttctttgatgactggccgagatcacttcaagaatcaggaaatgtagaaattaataataataataataataataataacaataacaataataggatgaggaagagtccaatgagctcagcttcaagcttatcaatttccataacgggaaacaactcatcatcaacttctgatatttcgttgaagttgtcaactggtggaaacggagaggaaatgggccacccagaagcccattctgatcgtcagcagcagtctcagttgggctgggcctcgagtggatgggcttcaaacccaatggcctcaatgggtgggccgctggctgaggccctgcgatcctccaactccaattcatcatctcctactagtgttctccatcagttgcctcctcgttcttctgcctctgacacttgcattgtcagcactgcggccgctgccatgcagcagcacctgatgcagatgcagcccatgatggcagcctattatcccaacaacgtcaccactgatcacattcaacagtatttggatgaaaacaagtcactgatcctgaagattgttgagagccagaattcaggaaaattgggtgaatgtgcagagaaccaagctaggctgcagcgaaacctcatgtacctggctgctattgctgattctcagccccagcctcctacaatgcattctcagtatccttcaagtagtattatgcagcctggagcacattacatgcaacagcaccaacaagctcagcagataactcaacaatcactcatggcgggaggctcgtccatgctgtacaaccagcagccatttacagctttgcaacagcaagcactgcacaaccagctcggcatgagctctggtggaaactcgggacttcacatgctgcaaagtgacatgggtagcgctggaggcagtgggcagcagcttggaggtggaggatttcctgatttcagccgtggctctggaggagaagggatgcatagaggaatggctggcggtagtaagcaagatatgggaagttctggatcggctgaaggccgaggagggagctctggaggtgacgggggtgaaaccctttacttaaaatctgccgatgatgggaattaagcttaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtg

SEQ ID NO: 6 - DNA, 20 bp, Cannabis sativa
  source 1..20: mol_type = genomic DNA
accaattccagaaactgcaa

SEQ ID NO: 7 - DNA, 20 bp, Cannabis sativa
  source 1..20: mol_type = genomic DNA
cccttacggtggtataatgg

SEQ ID NO: 8 - DNA, 180 bp, synthetic construct
  source 1..180: mol_type = other DNA
gtgatcaaaagtcccacatcgatcaggtgatatatagcagcttagtttatataatgatagagtcgacatagcgattgaccaattccagaaactgcaagttttagagctagaaatagcaagttaaaataaggctagtccgttatcaacttgaaaaagtggcaccgagtcggtgcttttttt

SEQ ID NO: 9 - DNA, 180 bp, synthetic construct
  source 1..180: mol_type = other DNA
gtgatcaaaagtcccacatcgatcaggtgatatatagcagcttagtttatataatgatagagtcgacatagcgattgcccttacggtggtataatgggttttagagctagaaatagcaagttaaaataaggctagtccgttatcaacttgaaaaagtggcaccgagtcggtgcttttttt

SEQ ID NO: 10 - DNA, 1641 bp, Cannabis sativa
  source 1..1641: mol_type = genomic DNA
atggatcattcttcttcttcttcatcaactccttatcatgacgatcaccatcatcaaaactggctagctttctctctctccaaccataaccctctctctctcttccaaacctccttcactactcccaataaccaagatcatcatcaagatgcttcatcaacggatctcgccatgttcaccggtgcagcaccgaagctggagaactttctcggcggaggacccaccaccgcctcctctccaaatagtaataataataatcagtttaacggcgttgatcaaacggcggtaacggaagctgatgagctgtatgagtcggagcttaagactattgccgctagctttctccatggtttcaactctcatataaataataataataataatattatgagtattattactaatgatagtaatctcagttgccagctggaagcgccgccgtctgcggtggctccggtgtcgtctaagaaaaccgttgacacttttggtcaacgtacctctatataccgtggtgttactcgacatagatggactggtagatatgaagctcatttgtgggacaacagttgccgaagagaaggccagagtagaaaagggcgacaagtttatttgggtggatatgataaagaagaaaaggcagcaagagcttatgatttggctgcccttaagtactggggtcctaccaccactacaaattttgcagtgtctaattacgaaaaagaattagaagatatgacgaacatgactaggcaagaattcgttgcttcacttcgaaggaaaagtagtggattttctagaggagcttcaatatacagaggcgtcacaaggcaccaccaacatggtcgatggcaggcaagaattggaagagtagcaggaaacaaagatctctaccttggcacctttagcacacaagaagaagcagccgaggcatacgacatcgcggcgataaaattccgaggcctaaacgccgtaacaaacttcgacatgagccgttacgacgttaaaagcatagccaactctaatctccccgttggaggaatgtcaaacaacaccaaactttccaaaacctcacccgaacgggcgattgacaacctatcatcgcccgcttcatcatccctcgtcgccttctcctcctcggccaccaccaacaacaacaacacaacaccccaacaacaacaacaacaacaaatgtcctccaatctaagctttactcttcccatcaaacaagacctaacaacaacgacaacatcgtcaacggattattggtcaaacattttcggtttccaaaaccctaaccctagtagtactactagtactactccttccttattgttgggccataatagtcacaacctctcggccacaacaactaatgcaacaacaactacaacaacaacaagtaatggagggtattatggtaatttcatcgagtcaatttctaataatagtaataataatactaacttgggttatggatcaggattaagtagctggattagtaatagtaatcataatattaacggagggagtagtaatagtaatagtaatgttcataatcatcttcatcatcatcatcatcatgaagttgttcatgcgaaacaacctagtctttttcaaacaccaatattcggcatggaataa

SEQ ID NO: 11 - protein, 546 aa, Cannabis sativa
MDHSSSSSSTPYHDDHHHQNWLAFSLSNHNPLSLFQTSFTTPNNQDHHQDASSTDLAMFTGAAPKLENFLGGGPTTASSPNSNNNNQFNGVDQTAVTEADELYESELKTIAASFLHGFNSHINNNNNNIMSIITNDSNLSCQLEAPPSAVAPVSSKKTVDTFGQRTSIYRGVTRHRWTGRYEAHLWDNSCRREGQSRKGRQVYLGGYDKEEKAARAYDLAALKYWGPTTTTNFAVSNYEKELEDMTNMTRQEFVASLRRKSSGFSRGASIYRGVTRHHQHGRWQARIGRVAGNKDLYLGTFSTQEEAAEAYDIAAIKFRGLNAVTNFDMSRYDVKSIANSNLPVGGMSNNTKLSKTSPERAIDNLSSPASSSLVAFSSSATTNNNNTTPQQQQQQQMSSNLSFTLPIKQDLTTTTTSSTDYWSNIFGFQNPNPSSTTSTTPSLLLGHNSHNLSATTTNATTTTTTTSNGGYYGNFIESISNNSNNNTNLGYGSGLSSWISNSNHNINGGSSNSNSNVHNHLHHHHHHEVVHAKQPSLFQTPIFGME

SEQ ID NO: 12 - DNA, 3574 bp, synthetic construct
  source 1..3574: mol_type = other DNA
gaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaagtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtcaagactggcgaacagttcatacagagtcttttacgactcaatgacaagaagaaaatcttcgtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataaggtaaatttctgtgttccttattctctcaaaatcttcgattttgttttcgttcgatcccaatttcgtatatgttctttggtttagattctgttaatcttagatcgaagatgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggagctcatttttacaacaattaccaacaacaacaaacaacaaacaacattacaattacatttacaattatcgatacaatggatcattcttcttcttcttcatcaactccttatcatgacgatcaccatcatcaaaactggctagctttctctctctccaaccataaccctctctctctcttccaaacctccttcactactcccaataaccaagatcatcatcaagatgcttcatcaacggatctcgccatgttcaccggtgcagcaccgaagctggagaactttctcggcggaggacccaccaccgcctcctctccaaatagtaataataataatcagtttaacggcgttgatcaaacggcggtaacggaagctgatgagctgtatgagtcggagcttaagactattgccgctagctttctccatggtttcaactctcatataaataataataataatattatgagtattattactaatgatagtaatctcagttgccagctggaagcgccgccgtctgcggtggctccggtgtcgtctaagaaaaccgttgacacttttggtcaacgtacctctatataccgtggtgttactcgacatagatggactggtagatatgaagctcatttgtgggacaacagttgccgaagagaaggccagagtagaaaagggcgacaagtttatttgggtggatatgataaagaagaaaaggcagcaagagcttatgatttggctgcccttaagtactggggtcctaccaccactacaaattttgcagtgtctaattacgaaaaagaattagaagatatgacgaacatgactaggcaggaattcgttgcttcacttcgaaggaaaagtagtggattttctagaggagcttcaatatacagaggcgtcacaaggcaccaccaacatggtcgatggcaggcaagaattggaagagtagcaggaaacaaagatctctaccttggcacctttagcacacaagaagaagcagccgaggcatacgacatcgcggcgataaaattccgaggcctaaacgccgtaacaaacttcgacatgagccgttacgacgttaaaagcatagccaactccaatctccccgttggaggaatgtcaaacaacaccaaactttccaaaacctcacccgaacgggcgattgacaacctatcatcgcccgcttcatcatccctcgtcgccttctcctcctcggccaccaccaacaacaacaacacaacaccccaacaacaacaacaacaaatctcctccaatctaagctttactctaccaatcaaacaagacctaacaacaacgacaacatcgtcaacggattattggtcaaacattttcggtttccaaaaccctaaccctagtagtactactagtactactccttccttattgttgggccataatagtcacaacctctcggccacaacaactaatgcaacaacaactacaacaacaacaagtaatggagggtattatggtaatttcatcgagtcaatttctaataatagtaataataatactaacttgggttatggatcaggattaagtagctggattagtaatagtaatcataatattaacggagggagtagtaatagtaataatgttcataatcatcttcatcatcatcatcatcatgaagttgttcatgcgaaacaacctagtctttttcaaacaccaatattcggcatggaataagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgct